(19) United States
(12) Patent Application Publication
(10) Pub. No.: US 2021/0257095 A1
(43) Pub. Date: Aug. 19, 2021

(54) MEDICAL MACHINE LEARNING SYSTEM AND METHOD

(71) Applicants: BAXTER INTERNATIONAL INC., Deerfield, IL (US); BAXTER HEALTHCARE SA, Glattpark (Opfikon) (CH)

(72) Inventor: John Ramez Zacharia, Chicago, IL (US)

(21) Appl. No.: 17/177,963

(22) Filed: Feb. 17, 2021

Related U.S. Application Data
(60) Provisional application No. 62/977,850, filed on Feb. 18, 2020.

(51) Int. Cl. G16H 50/20; G06N 20/00; G06N 5/02; G16H 50/70; G16H 50/30; G16H 40/20; G16H 15/00; G16H 10/60; G16H 40/67; G16H 40/40

(52) U.S. Cl. CPC G16H 50/20; G06N 20/00; G06N 5/027; G16H 50/70; G16H 40/40; G16H 40/20; G16H 15/00; G16H 10/60; G16H 40/67; G16H 50/30

(57) ABSTRACT

Methods and systems for providing renal-related clinical decision support are disclosed. In an example, a medical treatment system includes a plurality of medical machines located at each of a plurality of hospitals. At least one medical machine of each hospital generates machine output data. The medical treatment system also includes a plurality of sources of data external to the medical machines and a logic engine implemented on a computer. The logic engine is configured to obtain a module formed via data from the plurality of sources and from the machine output data. The module quantifies a risk assessment for an adverse health condition of a patient undergoing treatment by one of the medical machines. The logic engine is also configured to compare an outcome from the module to a clinical setpoint for the adverse health condition, and provide a notification based on the comparison.

### PRIORITY CLAIM

This application claims priority to and the benefit as a non-provisional application of U.S. Provisional Patent Application No. 62/977,850, filed Feb. 18, 2020, the entire contents of which are hereby incorporated by reference and relied upon.

### TECHNICAL FIELD

The present disclosure relates generally to medical machines, and in particular to a clinical decision support system that uses machine learning.

### BACKGROUND

Acute kidney injury (“AKI”) is fairly common but is under-recognized in hospital patients, especially in certain countries. It has been reported that worldwide, twenty percent of hospitalized patients have AKI. A larger number of intensive care unit (“ICU”) patients have AKI, where fifteen to twenty-five percent of such patients receive some form of renal replacement therapy (“RRT”). Approximately twenty-seven percent of pediatric and young adult ICU patients develop AKI during the first week after admission to the hospital.

AKI is associated with a nearly ten-fold increased risk of inpatient mortality. AKI is also associated with adverse long term outcomes, such as hypertension, chronic kidney disease, end-stage renal disease, and mortality. Major contributors to AKI include septic shock (˜47% of instances), major surgery (˜34% of instances), cardiogenic shock (˜27% of instances), hypovolemia (˜25% of instances), drug induced (˜19% of instances), hepatorenal syndrome (˜6% of instances), and obstructive uropathy (˜3% of instances).

In response to an increasing rate of AKI among hospitalized patients, coupled with concerns that AKI may be in some cases iatrogenic or improperly managed, the Centers for Medicare and Medicaid Services have proposed that AKI be added to a monitored list of inpatient harms that can be used to determine hospital reimbursement rates. In this environment, predicting AKI before it occurs and responding appropriately to AKI when it develops is an issue of high importance to patients, physicians, and healthcare systems alike.

Existing decision support models for detection of AKI are based upon tracking serum creatinine or urine biomarkers, each of which have resulted in inadequate improvements in mortality and length of hospital stay. An improved overall regime for predicting and enabling treatment of AKI is accordingly needed.

### SUMMARY

The system and method of the present disclosure include a software-based interface that enables clinicians to diagnose, predict, and prevent a number of renal related conditions in a critical care environment by making contextual recommendations using real-time clinicial data. The system and method, in an embodiment, enable the integration of a number of physiological, administrative, and/or device-based data streams in a critical care environment. The system and method use the data streams to continuously monitor and trigger alerts indicaitve of an onset of renal related conditions such as AKI. The disclosed system and method use appropriate rules or learning engines in an appropriate context to analyze the data streams for predicting the onset of renal related conditions. The system and method enable the development of diagnostic and predictive rules or learning engines that drive downstream flags, quantified risk scores, or risk clustering for target patients. The system and method also enable the use of the predefined logic engines to predict, diagnose, and/or recommend courses of actions for renal related conditions at a patient, unit, or hospital level. The system and method further provide for appropriate alert, intervention, or communication techniques via multiple delivery channels to ensure full care team awareness of the identifyed target issues. As used herein, “logic engine” refers to either or both a “rules engine” and/or a “learning engine”.

In one embodiment, a rules engine is driven by predetermined relationships between patient specific descriptors and corresponding rule or learning modules, which are determined at one or more server computer maintained by (or under the control of) a provider of the disclosed system. The one or more server computer is configured to access the data pool of each hospital. Upon analyzing the data pool of each hospital, the one or more server computer develops a rules engine for each hospital, wherein the rules engine may be the same or different for each hospital depending on clinical need, preference, and/or risk tolerance.

In another embodiment, a learning engine is developed using machine learning (“ML”) or artificial intelligence (“AI”) that improves the performance and functionality of the learning engine across diverse patient types. The ML/AI learning engine may be employed via any one or more learning model, such as: an artificial neural network, a Bayesian network, a decision tree, federated learning, a genetic algorithm, a support vector machine, and/or a training model. The models may be employed using any one or more of: reinforcement learning, self learning, supervised learning, and/or unsupervised learning.

The learning engine, in an embodiment, is pre-trained using representative data streams to achieve required performance levels prior to clinical implementation. Prior to clinical implementation and use, the learning engine can be locked in a pre-conditioned state and maintain currently provisioned algorithms. Alternatively, the learning engine can be unlocked to dynamically and continuously re-train using ongoing data throughput during clinical usage. The re-training configuration can be more quickly and accurately implemented into hospital environments by establishing universal core logic via pre-training, and then be optimized against unique clinical practices or patient conditions. The re-training configuration can also work to reduce the number of required data inputs to the universal learning engine based on limitations of local data systems while still maintaining an acceptable level of performance. The ML or AI features of the learning engine determine the appropriate prediction or diagnostic elements by viewing the available dataset against target results. Using a single or dynamic phase machine learning or appropriate AI techniques enables the learning module system to develop a set of learning modules that predict or detect results pertaining to adverse health conditions in real world settings.

In light of the disclosure herein and without limiting the disclosure in any way, in a first aspect of the present disclosure, which may be combined with any other aspect listed herein, a medical treatment system includes: a plurality of medical machines located at each of a plurality of hospitals, at least one medical machine of each hospital generating machine output data; a plurality of sources of data external to the medical treatment machines; and a logic engine implemented on a computer, the logic engine configured to (i) obtain a module formed via data from the plurality of sources and from the machine output data, the module quantifying a risk assessment for an adverse health condition of a patient undergoing treatment by one of the medical treatment machines, (ii) compare an outcome from the module to a clinical setpoint for the adverse health condition, and (iii) provide a notification based on the comparison.

In a second aspect of the present disclosure, which may be combined with any other aspect listed herein, the logic engine is a rules engine and the module is built on predetermined relationships between variables associated with the module.

In a third aspect of the present disclosure, which may be combined with any other aspect listed herein, the logic engine is a learning engine and the module determines an algorithm based on analyzed results.

In a fourth aspect of the present disclosure, which may be combined with any other aspect listed herein, a medical treatment system includes: a plurality of medical treatment machines; a plurality of sources of data external to the medical treatment machines; and a rules engine implemented on a computer, the rules engine configured to (i) obtain a module formed via data from the plurality of sources, the module quantifying a risk assessment for an adverse health condition of a patient undergoing treatment by one of the medical treatment machines, (ii) determine a result from the module, (iii) compare the result to a clinical setpoint for the adverse health condition, and (iv) provide a notification based on the comparison.

In a fifth aspect of the present disclosure, which may be combined with any other aspect listed herein, at least one of (a) the module is a first module and the adverse health condition is a first adverse health condition, and wherein the rules engine is further configured to repeat (i) to (iv) for a second module and a second adverse health condition, or (b) the patient is a first patient, and wherein the rules engine is further configured to repeat (i) to (iv) for a second patient.

In a sixth aspect of the present disclosure, which may be combined with any other aspect listed herein, the module is a regression module and the data forming the module includes data concerning at least one physiological condition of the patient.

In a seventh aspect of the present disclosure, which may be combined with any other aspect listed herein, the notification indicates that the patient is not at risk for the adverse health condition, is at risk for the adverse health condition, or is experiencing the adverse health condition.

In an eighth aspect of the present disclosure, which may be combined with any other aspect listed herein, the medical treatment system is configured to provide the notification from rules engine to an interface including at least one of a computer monitor, tablet, mobile device, or smartphone.

In a ninth aspect of the present disclosure, which may be combined with any other aspect listed herein, the plurality of medical treatment machines are located at multiple hospitals, and the plurality of sources of data external to the medical treatment machines includes data from electronic medical records of the multiple hospitals.

In a tenth aspect of the present disclosure, which may be combined with any other aspect listed herein, the module is formed external to the rules engine and is transferred to the rules engine or the module is formed by the rules engine.

In an eleventh aspect of the present disclosure, which may be combined with any other aspect listed herein, the clinical setpoint for the adverse health condition is standardized for all patients or customized for individual patients.

In a twelfth aspect of the present disclosure, which may be combined with any other aspect listed herein, the rules engine is configured to adapt the module over time based on new data from the plurality of sources.

In a thirteenth aspect of the present disclosure, which may be combined with any other aspect listed herein, the rules engine is configured to adapt the module over time to improve performance of the module.

In a fourteenth aspect of the present disclosure, which may be combined with any other aspect listed herein, the rules engine is configured to adapt the module over time based on a different module developed for a different medical treatment machine or a different patient.

In a fifteenth aspect of the present disclosure, which may be combined with any other aspect listed herein, a medical treatment system includes: a plurality of medical treatment machines; a plurality of sources of data external to the medical treatment machines; and a learning engine implemented on a computer, the learning engine configured to form a module quantifying a risk assessment for an adverse health condition of a patient undergoing treatment by one of the medical treatment machines, the learning engine configured to make an association between the adverse health condition and at least one factor associated with the data external to the medical treatment machines, the module including the at least one factor.

In a sixteenth aspect of the present disclosure, which may be combined with any other aspect listed herein, the learning engine employs at least one tool including a training tool, a preprocessing tool, a postprocessing tool, and a clinical decision support tool.

In a seventeenth aspect of the present disclosure, which may be combined with any other aspect listed herein, the learning engine is configured to retrain the module based upon clinical testing of the module.

In an eighteenth aspect of the present disclosure, which may be combined with any other aspect listed herein, the learning engine is configured to adapt the module over time based on new data from the plurality of sources.

In a nineteenth aspect of the present disclosure, which may be combined with any other aspect listed herein, the learning engine is configured to adapt the module over time to improve performance of the module.

In a twentieth aspect of the present disclosure, which may be combined with any other aspect listed herein, the at least one factor is determined empirically.

In a twenty-first aspect of the present disclosure, any of the structure and functionality disclosed in connection with FIG. 1 may be combined with any of the other structure and functionality disclosed in connection with FIG. 2.

In light of the present disclosure and the above aspects, it is therefore an advantage of the present disclosure to provide a system and associated method for predicting adverse health conditions associated with renal failure therapy treatments, including any associated intravenous drug delivery.

It is another advantage of the present disclosure to provide a system and associated method that is useable in different medical settings having varying sizes and capabilities.

It is a further advantage of the present disclosure to provide a system and associated method that may be self-adapting upon receiving new data.

It is still another advantage of the present disclosure to provide a system and associated method that may be self-adapting to improve performance.

It is still a further advantage of the present disclosure to provide a system and associated method that inputs data from many different sources.

It is yet another advantage of the present disclosure to provide a system and associated method that does not add a net positive workload to a clinician or other user.

Additional features and advantages are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein and it is expressly contemplated to claim individual advantageous embodiments separately. Moreover, it should be noted that the language used in the specification has been selected principally for readability and instructional purposes, and not to limit the scope of the inventive subject matter.

### BRIEF DESCRIPTION OF THE FIGURES

FIG. 1 is a schematic view of one embodiment for a medical machine learning system and associated method of the present disclosure.

FIG. 2 is a schematic view of a second embodiment for a medical machine learning system and associated method of the present disclosure.

### DETAILED DESCRIPTION

### Data Acquisition

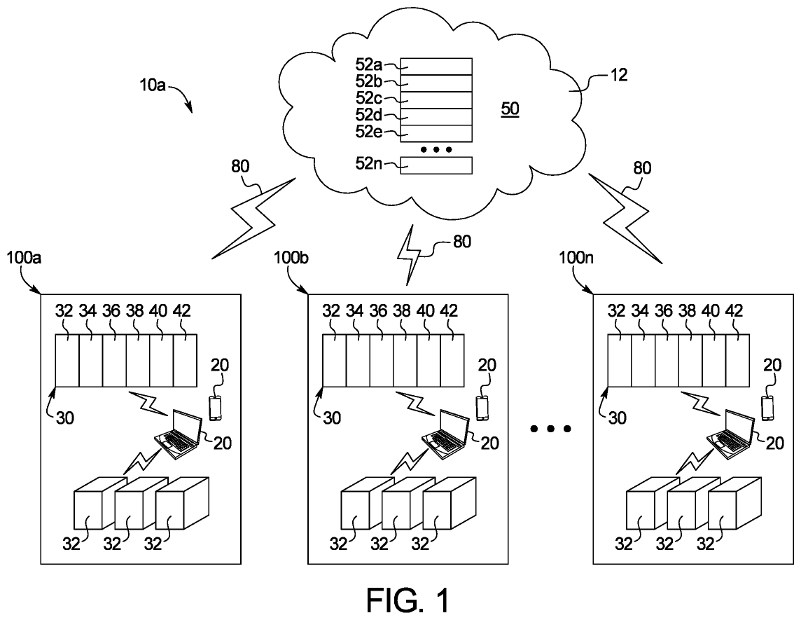

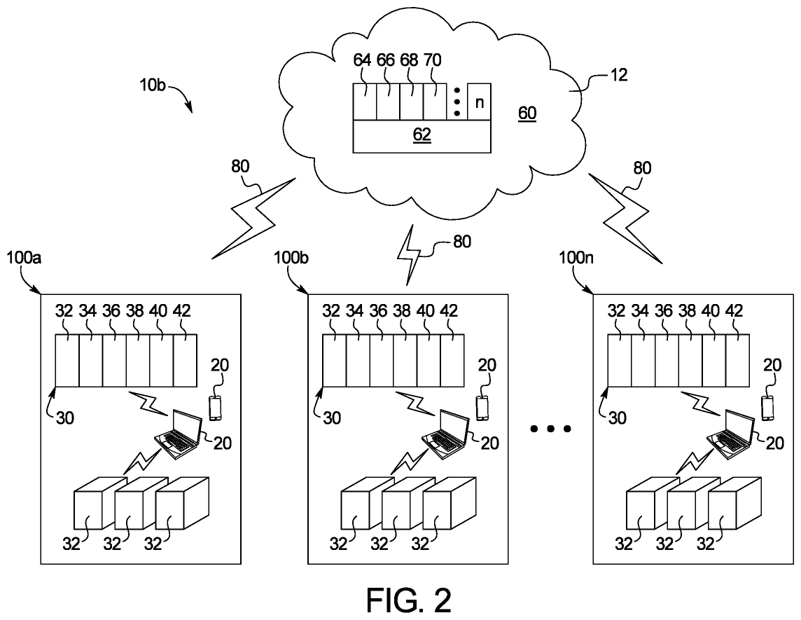

Referring now to the drawings and in particular to FIGS. 1 and 2, systems 10a and 10b and associated methods of the present disclosure each include a software-based interface 20 that enables clinicians and other caregivers or users to diagnose, predict, and prevent a number of renal related conditions in a critical care environment by making contextual recommendations using real-time patient data. Systems 10a and 10b enable the integration of a number of automated, manual, or device-based data streams in a critical care environment to continuously monitor and trigger appropriate rule or learning modules in an appropriate context.

Systems 10a and 10b each include a data pool 30 or “lake”, which is created in each hospital 100a to 100n. Data pool or lake 30 houses real time information that is unique and specific to a target patient. Data pool 30 includes multiple sources for contributing data. One source of information for the data pool is from one or more medical treatment machine 32, such as a continuous renal replacement therapy (“CRRT”) machine, an intermittent hemodialysis (“IHD”) machine, an infusion pump, a ventilator, diagnostic monitors, patient sensors, hospital bed settings, blood pressure systems, hemodynamic monitors, or patient weight scales, for example. Machine 32 may be present in the hospital room or may be a home-based machine.

Another source of information for the data pool 30 is hospital specific patient data 34. Hospital specific patient data 34 may include any one or more of laboratory results, qualitative assessments, manual clinician evaluations, risk scores (such as the Acute Physiology and Chronic Health Disease Classification (“APACHE”) risk score or the Sequential Organ Failure Assessment (“SOFA”) risk score), data concerning recent or upcoming procedures (such as surgery), or recent, concurrent, or upcoming regimens (such as antibiotic regimens), and/or non-traditional clinical sources such as social determinant data, for example.

A further source of information for the data pool 30 is natural language data 36, which may be provided via natural language processing, such as clinician notes, consultation requests, verbal dictation, and patient statements, for example. Natural language data 36 in an embodiment is entered into systems 10a and 10b via wired or wireless communication with interface 20, wherein a user (clinician, doctor, nurse or other caregiver) enters natural language data 36. It should be appreciated that data communication between any two or more components of systems 10a and 10b may be wired or wireless. Wired communication may be via USB, serial, or an Ethernet connection, for example. Wireless communication may be performed via any of Bluetooth™, Wi-Fi™, Zigbee®, Z-Wave®, wireless Universal Serial Bus (“USB”), Wi-Fi Direct, infrared protocols, or via any other suitable wireless communication technology.

Still another source of information for the data pool 30 is general administrative data 38, such as intake and discharge data, previous admission data, date of admission, age, gender, and basic profiling data, for example. General administrative data 38, in an embodiment, is entered into systems 10a and 10b via wired or wireless communication with a hospital's emergency medical record (“EMR”) database. General administrative data 38 is entered into an EMR database by hospital staff.

Still a further source of information for the data pool 30 is social data 40, such as non-traditional clinical data sources such as social determinants, social media posting frequency, natural language processing triggers, vocabulary scoring, professional standing, and changes in employment, for example. Social data 40, in an embodiment, is extracted from one or more social media source via a user of system 10a or 10b and entered into interface 20, which enters same wired or wirelessly to systems 10a and 10b.

Yet another source of information for the data pool 30 are large reference data sources 42 for comparative or benchmarking data, such as the ministry of health, payer sources, information health exchange, device manufacturer databases, and hospital networks, for example. Information from large reference data sources 42 is, in an embodiment, extracted from one or more of such sources 42 via a user of system 10a or 10b and entered into interface 20, which enters same wired or wirelessly to systems 10a and 10b. Alternatively, as illustrated in FIGS. 1 and 2, information from one ore more of large reference data sources 42 is entered into systems 10a and 10b directly via wired or wireless communication.

Systems 10a and 10b are configured to collect data from data sources 32 to 42 in real time or near real time for each patient and data pool 30. Systems 10a and 10b process and reduce the data from data sources 32 to 42 into an appropriate data warehousing or storage format, and making the combined data of data pool 30 available for the logic engines and modules discussed next. Data from data sources 32 to 42 may arrive in any one or more format, such as Extensible Markup Language (“XML”), JavaScript Object Notation (“JSON”), Fast Healthcare Interoperability Resource (“FHIR”) Health-Level Seven (“HL7”), or be a device specific data type. Systems 10a and 10b, in an embodiment, convert the different formats of data into one or more desired format for the logic engines and modules.

### Development of Logic Engines

Systems 10a and 10b provide for the development of diagnostic and predictive logic engines that drive downstream flags, risk categories, and/or quantified risk scores for target patients, for example. Systems 10a and 10b illustrate two possible approaches to the development of logic engines and modules that process the massaged data of data pools 30. System 10a of FIG. 1 illustrates a first approach, which includes a rules engine 50 that is driven by predetermined relationships between patient specific variables and corresponding rule modules, which are determined at one or more server computer 12, e.g., cloud server, maintained by (or under the control of) the provider of system 10a. One or more server computer 12 is able to access the data pool 30 of each hospital 100a to 100n. Upon analyzing the data pool 30 of each hospital 100a to 100n, one or more server computer 12 develops a predefined (non-learning) rules engine 50 for each hospital. Rules engine 50 may be the same or different for each hospital 100a to 100n.

In an embodiment, rules engine 50 for each hospital 100a to 100n of system 10a includes a plurality of rule modules 52a to 52n. Each rule module 52a to 52n is dedicated to making a risk assessment for a different adverse health condition for a patient. For example, one rule module 52a may be dedicated to determining the risk of AKI (first adverse health condition), while a second rule module 52b may be dedicated to determining a risk of nephrotoxic regimen (second, different adverse health condition).

Rule modules 52a to 52n of rules engine 50, in an embodiment, include a weighted regression model that quantifies a result or risk assessment for each adverse health condition. Taking AKI as an example adverse health condition, the rule module 52a may include an algorithm utilizing serum creatinine, cardiac risk scoring, patient age, patient gender, and blood pressure over time, which outputs a result for a patient. For that adverse health condition, the rule module 52a compares the result to a clinical setpoint, and outputs a patient status, which may indicate that the patient is not at risk for the particular health condition or trigger a flag, notification, alert, and/or alarm if the patient is at risk for, or experiencing, the particular health condition. In this example, AKI is the ultimate determinant as to whether the flag, notification, alert, and/or alarm is triggered. Rule modules 52a to 52n of rules engine 50 are, in an embodiment, established once and may be applied to all patients or substantially all of the patients. To provide some variability within a rule module 52a to 52n for different patients, it is contemplated for the clinical setpoint of the rule module to be varied based upon patient characteristics, such as physiological characteristics, including but not limited to age, sex, weight, body mass index, characteristic blood pressure, and the like.

It is also contemplated to build quality control and quality assurance into the rule modules 52a to 52n. In an embodiment, each regression model is provided as a recommendation. System 10a enables the clinicians or users, if desired, to access the rule modules 52a to 52n for each recommendation in real time to view the inputs and rules applied leading to the recommendation. For example, a “see risk evaluation” button may be provided on the clinician's or user's screen adjacent to the recommendation. When the clinician or user selects the “see risk evaluation” button, a screen or partial screen appears showing the regression model or other type of non-learning algorithm used to determine the risk evaluation. The user or clinician can review the inputs or variables involved and/or the weighting assigned to each. If the clinician or user disagrees with any one or more of the inputs and/or weighting, the clinician or user can ignore or place less emphasis on the risk evaluation and any associated recommendation, which provides a quality control feature to system 10a.

System 10a may also be configured to enable the clinician or user to revise the non-learning algorithm if the clinician or user feels confident that a change is needed, e.g., to remove or add an input and/or change a weight associated with the input. System 10a may be configured to then update the non-learning algorithm for (i) the associated clinician or user only, (ii) for all clinician's or users of the associated hospital 100a to 100n, or (iii) for all hospitals 100a to 100n of system 10a. In an alternative embodiment, system 10a does not automatically update the non-learning algorithm but instead publishes the proposed change for peer review. Based upon a peer review or other evaluation, e.g., clinician or user vote, advisory board decision, clinical evaluation, system 10a updates or does not update the non-learning algorithm based on the clinician's or user's suggested change(s). Any of the above implementations, however, provides a quality assurance feature to system 10a.

In an embodiment, the regression model of rule modules 52a to 52n of rules engine 50 is based on prevailing clinical expertise to select appropriate variables. The regression model may then be tested using the variables in a clinical environment to confirm accuracy and precision, and/or to modify as needed. An example regression model for rule modules 52a to 52n is illustrated for example via rule module 52c as follows:

regression model for rule module 52c: x₁+x₂+(0.5)x₃+(1.1)x₄=result,
where the result may be, for example, a number out of 100, and where
x₁ is hyperkalemia,
x₂ is acidaemia,
x₃ is pulmonary oedema, and
x₄ is uraemic complications.
An amount of any one of inputs x₁ to x₄ that is found to be present in the patient is converted (e.g., normalized) for implementation in rule module 52c and stored as converted into data pool 30 of the patient's hospital 100a to 100n. Rule module 52c (or rules engine 50 operating rule module 52c) then computes the result by inserting the converted number for any of x₁ to x₄ into the regression model. Suppose the result of the calculation is 88/100. Rule module 52c (or rules engine 50 operating rule module 52c) then compares the result to a clinical setpoint, which may be standardized for all patients or individualized for each patient, as discussed above. The use of rules engine 50 and its rule modules 52a to 52n is discussed in more detail below.

System 10a, in an embodiment, provides for intermediate modules that are configured to generate intermediate meta-variables between the structured data pool and the key risk determinations. The meta-variables are achieved using multiple raw or structured data sources to create a derived (meta) variable. The meta-variables are used to improve real-time performance by not having to wait for all data to be received to run the final risk score, but running periodically to maximize computer resources or simplify unconventional data (e.g., natural language processing, social media data, etc.). In the example of rule module 52c, any of x₁ to x₄ may be derived from an intermediate module prior to the overall calculation of the result of rule module 52c. Any of x₁ to x₄ may themselves be determined from a non-learning algorithm or be measured or entered data.

It should be appreciated from the example regression model above for rule module 52c that the result of the regression model does not have to represent a known health condition. The result may instead encompass multiple health conditions that combine to indicate an overall health status for a patient. The result may further alternatively encompass multiple factors that lead to a customized health condition developed specifically for a patient and/or for a hospital or a general criticality score to motivate clinical focus without recommending specific action. The result may of course represent a known health condition, such as AKI.

It should also be appreciated that a regression model, such as rule module 52c, is one example of a non-learning type of algorithm that may be used in connection with system 10a. Other non-leaning types of algorithms include partial least squares, principal component regression, and optimization techniques. Different non-learning algorithms may be used for different rule modules 52a to 52n.

### Learning Engines

FIG. 2 for system 10b illustrates a second approach, which includes a learning engine 60. In the second approach, learning engine 60 uses machine learning (“ML”) or artificial intelligence (“AI”) that improves the functionality of the logic engine across diverse patient types and improves a time to prediction. Where rules engine 50 includes predetermined or human determined algorithms, learning engine 60 is configured for the algorithms to be formed or computed based on a known outcome (adverse health condition) and data inputted. Learning engine 60 is dynamically trained and re-trained on datasets against specific patient conditions without prescribing a predictive formula. The ML or AI feature determines the appropriate prediction or diagnostic elements by analyzing the available dataset. Using machine learning or appropriate AI techniques enables system 10b to develop a set of learning modules 62a to 62n, which predict or detect results pertaining to adverse health conditions.

The ML or AI learning engine 60, in an embodiment, operates across multiple hospitals 100a to 100n, which if taken individually may not generate enough data for the learning engine 60. Different hospitals 100a to 100n, perhaps located worldwide, may work with different available data per patient. Taking the different hospitals 100a to 100n into account enables all, or an accumulated amount of, relevant data to be entered into learning engine 60. Taking the different hospitals 100a to 100n into account also helps ensure model performance at smaller or less busy hospitals that generate less data and that may have less bandwidth, processing power, and/or digital expertise. It should be appreciated however that any of hospitals 100a to 100n may generate enough data on its own to satisfy learning engine 60.

In an embodiment, ML or AI learning engine 60 includes at least one of the following characteristics:
1. Structure—learning engine 60 is formed from one of or a mixture of neutral networks, decision trees, support vector machines, and/or other ML or AI development approaches, which taken together, form a plurality of learning modules 62a to 62n. Learning modules 62a to 62n are predictors of adverse conditions, just as rules modules 52a to 52n. The primary difference being that the algorithms for rules modules 52a to 52n are based on defined relations, while the algorithms for learning modules 62a to 62n are artificially or machined developed. The structure of learning engine 60 in the illustrated embodiment of FIG. 2 is provided at cloud server 12. The structure includes a ‘baseline’ level 64 of stored information formed from the large, universal data pool 30. Hospitals and other users may install baseline level 64 initially and may not have to make any changes to it after installation. Baseline level 64 may or may not be altered by the re-training described below. Baseline level 64 may include an untouchable core, but may in certain instances, e.g., in the face of large data sets of contradictory clinical data, be re-trained. The structure of learning engine 60 also includes a plurality of learning engine tools 66, 68, 70 and 72, discussed next. Baseline level 64 and learning engine tools 66, 68, 70 and 72 form a decision support system of learning engine 60. Any one or more of tools 66, 68, 70 and 72 may be adapted for use with the rules engine 50 of system 10a.
2. Training/Re-training—ML or AI learning engine 60, in one embodiment, may provide a training tool 66 that enables hospitals to customize the baseline level 64 by updating or retraining the baseline level 64 using the hospital's specific data set. Such customization or re-training can either adjust or replace the previous baseline level 64 using the local data set. In this manner, learning engine 60 is configured to dynamically update its functionality at a hospital or regional level. The customization or re-training may be driven, for example, by one or more of (i) a different type of patient group or cohort (say, urban versus rural) that reduces the accuracy of the model on a new type of patient unless updated, (ii) a more limited available data set, such as a hospital only having five of a required twelve variables in an example ML or AI learning engine 60, or (iii) a substantially different sample frequency, e.g., one or more variable of the ML or AI engine is sampled only once a day vs twelve times a day in other hospitals. Any such variance, or a different variance, may require or prompt customization or re-training of baseline level 64 to improve the precision of the output for the previous baseline level and learning engine 60 accordingly.
   The training process of system 10b is unique in that the baseline level 64 of each hospital 100a to 100n contributing to ML or AI learning engine 60 is trained or re-trained independently based on an available operational dataset for the consituent hospital. Such a phased approach enables segmentation of available features based on accuracy, customer need, and available processing power.
   The training and re-training of training tool 66 is separated, in an embodiment, into (i) pre-training that uses representative data streams to achieve required performance levels prior to clinical implementation, and (ii) re-training that occurs after clinical implementation. Prior to clinical implementation and use, system 10b can configure training tool 66 to be either locked in the pre-trained state to maintain its current algorithm, or be unlocked to dynamically and continuously re-train using ongoing data throughput during clinical usage. The re-training configuration of training tool 66 can be more quickly and accurately implemented into hospital environments by establishing universal core logic via pre-training and then be optimized against unique clinical practices or patient conditions. The re-training configuration of tool 66 can also reduce the number of required data inputs to the universal learning engine based on limitations of local data systems while still maintaining an acceptable level of performance. The ML or AI features of the learning engine 60 determine the appropriate prediction or diagnostic elements by viewing the available dataset against target results. Using a single or dynamic phase machine learning or appropriate AI techniques enables the learning engine 60 to develop a set of learning modules 62a to 62n that predict or detect results pertaining to adverse health conditions in real world settings.
3. Input Data Preparation—ML or AI learning engine 60 includes a preprocessing tool 68 that is customized for the data streams of each particular hospital 100a to 100n. Preprocessing tool 68, in an embodiment, normalizes, standardizes, pre-treats, and/or removes outliers of quantitative or qualitative data to ensure model performance and accuracy. Preprocessing tool 68 may also remove common and/or unnecessary words such as, “it”, “the”, and the like, from natural language data to reduce a processing burden. Preprocessing tool 68 may further batch and reduce large data streams into a representative average or proxy value to likewise reduce a processing burden and improve time to prediction.
4. Postprocessing—the output of ML or AI learning engine 60 may also include a postprocessing tool 70, which in an embodiment, is also customized for each particular hospital's learning modules 62a to 62n. Postprocessing tool 70, in an embodiment, averages, normalizes, and/or aggregates learning engine 60 output to optimize for accuracy, e.g., against a target clinical condition.
5. Execution—The learning tools 66, 68 and 70 for each hospital 100a to 100n are assembled in an embodiment by a clinical decision support tool 72 for the execution phase of ML or AI learning engine 60. Support tool 72 manages outputs, and processes computational exceptions. Support tool 72 may employ usage criteria, e.g., via triggering a binary flag, a quantified result (which in itself may trigger another binary flag), and/or a direction indicator. In an embodiment, clinical decision support tool 72 is separate from training tool 66.

Training tool 66 of learning engine 60 may employ any one or more learning model to arrive at learning modules 62a to 62n, such as: an artificial neural network, a Bayesian network, a decision tree, federated learning, a genetic algorithm, a support vector machine, and/or a training model. The models may be employed using any one or more of: reinforcement learning, self learning, supervised learning or unsupervised learning. Given the breadth of certain diagnoses, it is likely that a single learning module 62a to 62n will not be able to accurately predict a clinical result. It is more likely that learning modules 62a to 62n are combined or built over time to produce a multifactored module, like module 52c discussed above, that accurately predicts a clinical result or adverse patient condition.

In an example, suppose training tool 66 of learning engine 60 learns that for a patient suffering from an adverse patient condition A, 100% of similar patients have factor x₁, 60% of the similar patients have factor x₂, 40% of the similar patients have factor x₃, and so on to xₙ as the percentages drop. Suppose that adding the percentages of all the presently learned correlations yields a total of 250%. Prior to clinical feedback, an arbitrary threshold percentage such as 80% may be set, such that any patient having a combined x₁+x₂+x₃+xₙ percentage score that is at least 80% of 250% (i.e., 200% or more) is deemed to be at risk of the patient condition A. The system 10b then causes an alert to be communicated to the patient or caregiver, so that the patient is then tested for adverse patient condition A. The re-training is then applied as the clinical testing yields feedback. In an embodiment, if greater than 80% of the patients tested are found to suffer from adverse patient condition A, then the arbitrary threshold percentage can be lowered, for example, until the actual perentage of people having adverse patient condition A meets the original threshold, e.g., 80%, where the original threshold is deemed to be the threshold at which it is worth the effort of clinical testing.

If clinical testing results in only a small number of actual patients having adverse patient condition A, then the arbitrary threshold can be raised until the clinical testing results in a minimum threshold, e.g., at least half, of the actual patients having adverse condition A. If the minimum threshold cannot be met, then the algorithm x₁+x₂+x₃+xₙ can be modified and re-tested (retrained) or scrapped. But importantly, as training tool 66 of learning engine 60 correlates different factors to patients having patient condition A, algorithm x₁+x₂+x₃+xₙ can be added to or modified to include the newly correlated one or more factor.

### Use of Rules Engine and Leaning Engine

Systems 10a and 10b allow for the use of rules engine 50 and ML or AI learning engine 60, respectively, to predict, diagnose, and/or recommend courses of actions for renal related or other conditions at a patient, machine 32, and/or hospital level. Each learning module 62a to 62n of learning engine 60 may include a separate clinical ‘decision’ or ‘pre-decision’ that is used as a trigger for a notification to a user (e.g., recommendation to a physician or clinician), as input for another learning module(s), or as an input to a medical machine 32 (e.g., as a notification on a display device of the medical machine). Engines 50 and 60, in an embodiment, execute based on real-time input of patient related data streams for the following clinical states for current or proposed treatment regimines. The states may be a current or predicted future status based on current or proposed treatment regimines. Example states include, but are not limited to:

current renal health/injury state, quantified (score/level), and/or directional (up or down) from a previous state,

quantified or directional assessment of a renal health score and anticipated future trends,

quantification of changes in fluid status, hyper or hypovolemic states,

AKI presence and stage status,

lack of AKI or related risks,

hemodynamic instability and trends,

pharmaceutical evaluation, including risk of nephrotoxic regimen,

vasopressors and blood pressure state,

fluid dosing/net balance status and history, and

CRRT performance and prescription requirements.

Rules and learning engines 50 and 60 evaluate, at a hospital or unit level, the above metrics or states using different acceptance criteria, which is sufficient to aggregate unit level risk to alert clinicians or other users of engines 50 and 60 to review their approach periodically, e.g., over the next three to five days, even if an individual risk level of each patient does not warrant such a change. Based on the states detected above, engines 50 and 60 may output to clinicians, or other users of the logic engine, additional logic layers to provide appropriate contextual information to the users to build corresponding treatment bundle recommendations for high probability state or predictions. The additional logic layers include but are not limited to:

additional diagnostics,

expanded care team support or outside consultation,

interventional models, such as fordiuretics, alteration of pharmaceutical approach, or dialysis,

step down/alternative therapy, and

recommendations for post acute care transitions and tracking for chronic renal diseases.

### Alterts and Intervention

Rules and learning engines 50 and 60 also provide appropriate alert, intervention, and/or communication techniques via one or more delivery channel 80 (wired or wireless) to ensure another system, clinician, or other user has awareness of any patient issue. Delivery channel 80 delivers any one or more of an audio, visual, or audiovisual alert or notification provided at any one or more of interface 20 (e.g., doctor or clinician computer monitor, tablet, or mobile device, e.g., smartphone) and/or medical machine 32. The alerts or notifications may signal, without limitation, that the patient is not at risk for an adverse health condition, is at risk for the adverse health condition, or is experiencing the adverse health condition.

A major factor in the utility of systems 10a and 10b of the present disclosure is to provide a system that fits into existing clinical workflows. It is a goal of systems 10a and 10b not to add net positive work to the clinician, so that the chances of its utilization are high. Systems 10a and 10b may accordingly provide, but are not limited to, the following contextual indicators:
systems 10a and 10b may present information within a dashboard on a suitable interface 20, such as a computer monitor, tablet, or mobile device, e.g., smartphone;
systems 10a and 10b may present information corresponding to individual patient status based on urgency, timeliness, and hospital preference;
alerts and notifications for the systems 10a and 10b are, in an embodiment, contextual and may describe any one or more of key criteria or alert levels including a current state of the patient, an anticipated future state, and/or a recommended course of action;
notification routing for the systems 10a and 10b may be based upon urgency of assessment or rule module or learning module type, where higher urgency levels may be routed to mobile devices 20 or group chat programs, while lower level alerts are displayed locally or perhaps not at all; and
underlying data, such as the key inputs used to trigger an associated rule or learning module, may be mirrored or presented in an alert to help motivate clinician action.

It should be understood that various changes and modifications to the presently preferred embodiments described herein will be apparent to those skilled in the art. Such changes and modifications can be made without departing from the spirit and scope of the present subject matter and without diminishing its intended advantages. It is therefore intended that such changes and modifications be covered by the appended claims.

The invention is claimed as follows:

1. A medical treatment system comprising:
a plurality of medical machines located at each of a plurality of hospitals, at least one medical machine of each hospital generating machine output data;
a plurality of sources of data external to the medical machines; and
a logic engine implemented on a computer, the logic engine configured to
(i) obtain a module formed via data from the plurality of sources and from the machine output data, the module quantifying a risk assessment for an adverse health condition of a patient undergoing treatment by one of the medical machines,
(ii) compare an outcome from the module to a clinical setpoint for the adverse health condition, and
(iii) provide a notification based on the comparison.

2. The medical treatment system of claim 1, wherein the logic engine is a rules engine and the module is built on predetermined relationships between variables associated with the module.

3. The medical treatment system of claim 1, wherein the logic engine is a learning engine and the module determines an algorithm based on analyzed results.

4. A medical treatment system comprising:
a plurality of medical treatment machines;
a plurality of sources of data external to the medical treatment machines; and
a rules engine implemented on a computer, the rules engine configured to
(i) obtain a module formed via data from the plurality of sources, the module quantifying a risk assessment for an adverse health condition of a patient undergoing treatment by one of the medical treatment machines,
(ii) determine a result from the module,
(iii) compare the result to a clinical setpoint for the adverse health condition, and
(iv) provide a notification based on the comparison.

5. The medical treatment system of claim 4, wherein at least one of (a) the module is a first module and the adverse health condition is a first adverse health condition, and wherein the rules engine is further configured to repeat (i) to (iv) for a second module and a second adverse health condition, or (b) the patient is a first patient, and wherein the rules engine is further configured to repeat (i) to (iv) for a second patient.

6. The medical treatment system of claim 4, wherein the module is a regression module and the data forming the module includes data concerning at least one physiological condition of the patient.

7. The medical treatment system of claim 4, wherein the notification indicates that the patient is not at risk for the adverse health condition, is at risk for the adverse health condition, or is experiencing the adverse health condition.

8. The medical treatment system of claim 4, which is configured to provide the notification from the rules engine to an interface including at least one of a computer monitor, a tablet, a mobile device, or a smartphone.

9. The medical treatment system of claim 4, wherein the plurality of medical treatment machines are located at multiple hospitals, and the plurality of sources of data external to the medical treatment machines includes data from electronic medical records of the multiple hospitals.

10. The medical treatment system of claim 4, wherein the module is formed external to the rules engine and is transferred to the rules engine or the module is formed by the rules engine.

11. The medical treatment system of claim 4, wherein the clinical setpoint for the adverse health condition is standardized for all patients or customized for individual patients.

12. The medical treatment system of claim 4, wherein the rules engine is configured to adapt the module over time based on new data from the plurality of sources.

13. The medical treatment system of claim 4, wherein the rules engine is configured to adapt the module over time to improve performance of the module.

14. The medical treatment system of claim 4, wherein the rules engine is configured to adapt the module over time based on a different module developed for a different medical treatment machine or a different patient.

15. A medical treatment system comprising:
a plurality of medical treatment machines;
a plurality of sources of data external to the medical treatment machines; and
a learning engine implemented on a computer, the learning engine configured to form a module quantifying a risk assessment for an adverse health condition of a patient undergoing treatment by one of the medical treatment machines, the learning engine configured to make an association between the adverse health condition and at least one factor associated with the data external to the medical treatment machines, the module including the at least one factor.

16. The medical treatment system of claim 15, wherein the learning engine employs at least one tool including a training tool, a preprocessing tool, a postprocessing tool, and a clinical decision support tool.

17. The medical treatment system of claim 15, wherein the learning engine is configured to retrain the module based upon clinical testing of the module.

18. The medical treatment system of claim 15, wherein the learning engine is configured to adapt the module over time based on new data from the plurality of sources.

19. The medical treatment system of claim 15, wherein the learning engine is configured to adapt the module over time to improve performance of the module.

20. The medical treatment system of claim 15, wherein the at least one factor is determined empirically.
